SEQUENCE LISTING (3 sequences)

SEQ ID NO: 1 - protein, 11 aa, Homo sapiens
ALQYKLEGTTR

SEQ ID NO: 2 - protein, 11 aa, Homo sapiens
RLTRKRGLKLA

SEQ ID NO: 3 - protein, 11 aa, Homo sapiens
ALSLSNKFVEG

(19) United States
(12) Patent Application Publication
(10) Pub. No.: US 2003/0157567 A1
(43) Pub. Date: Aug. 21, 2003

(54) Oxidised fragments of apolipoprotein B and their use

(76) Inventors: Joe Lunec, West Midlands (GB); Ruth Bevan, West Bridgford (GB); Helen Griffiths, Solihull (GB)

Correspondence Address: MONIQUE A. MORNEAULT, 311 S. WACKER DRIVE, 53RD FLOOR, CHICAGO, IL, 60606-6622, US

(21) Appl. No.: 10/358,594

(22) Filed: Feb. 5, 2003

Related U.S. Application Data
(63) Continuation of application No. 09/398,902, filed on Sep. 20, 1999.

(51) Int. Cl. G01N 33/53; C07K 16/18; C07K 14/775

(52) U.S. Cl. 435/7.1; 530/388.25; 530/359

(57) ABSTRACT

There is disclosed a molecule having the sequence of SEQ ID NO: 1 or a partially modified form thereof or an analogue thereof, lysine 5 being conjugated with MDA, and which inhibits uptake by the high affinity LDL receptor of LDL or a partially modified form thereof.

Atherosclerosis is a major cause of mortality in all western populations. High serum cholesterol levels are associated with increased cardiovascular risk (Marmot, M., 1988, Atherosclerosis Reviews, 18: 95-108; WHO Monica Project, 1988, World Health Statistics Quarterly, 41: 115-138). However, oxidised sterols (cholesterol being a sterol) and not cholesterol per se appear to be the true agents which induce atherosclerotic lesions. Low levels of antioxidants, particularly vitamin E and B carotene are also associated with an increased risk of cardiovascular disease (Diplock, A. T., 1994, Mol. Asp. Med., 115: 295-376). These data suggest a role for the oxidation of lipids in the aetiopathogenesis of atherosclerosis.

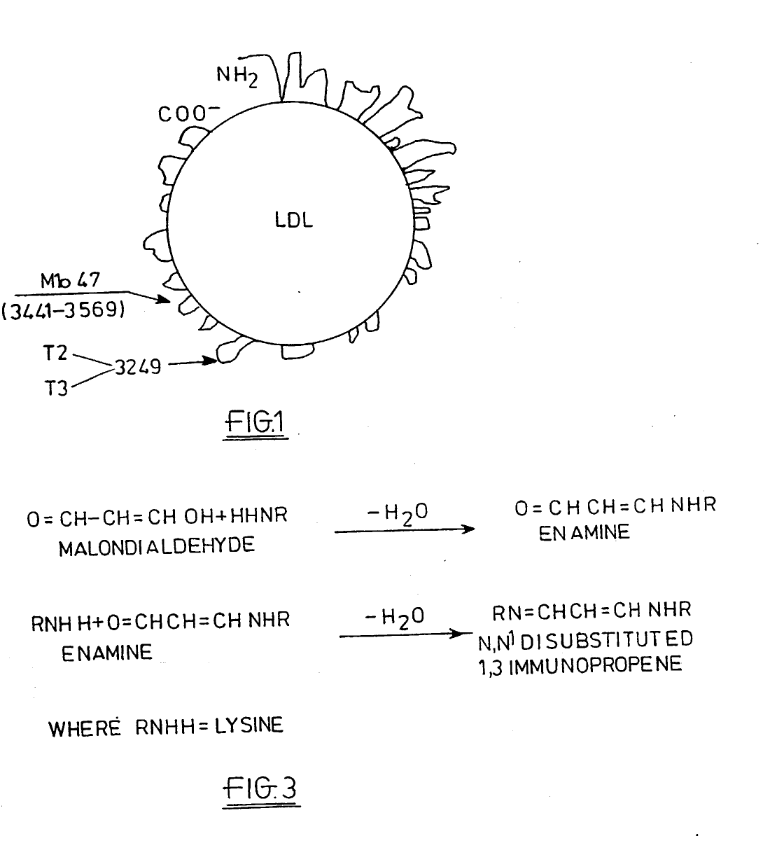

Circulating cholesterol is contained primarily in apoprotein B-based low density lipoprotein (LDL)—a spheroidal particle comprising approximately 1500 cholesterol ester molecules surrounded by a layer of 800 phospholipid molecules, 500 cholesterol molecules and at least one 550 kDa molecule of apoprotein B100 (Apo B) (FIG. 1). Increased levels of LDL correlate strongly with accelerated atherosclerosis. Atherosclerosis is characterised by thickening and degeneration of the arterial intima, the pathogenesis falling into two defined stages—a first stage in which fatty streaks containing foam cells form in the intima, and a second stage in which fibrous plaques are formed within the artery.

Foam cells are formed from macrophages when oxidised LDL is endocytosed by the macrophages via a scavenger receptor. This fools the cell into believing that it has taken up too little cholesterol, causing cholesterol to enter the cell via the high affinity LDL receptors (which recognise predominantly the Apo B portion of LDL) in an uncontrolled manner (Steinberg, D. et al., 1989, N. Eng. J. Med., 320 (14): 915-924; Goldstein, J. L. et al., 1979, Proc. Natl. Acad. Sci. USA., 76 (1): 333-337) instead of its usual precisely controlled manner. This saturation of the macrophage with cholesterol results in its morphological change into a foam cell.

The oxidation of LDL results in the covalent binding of the oxidation products of the fatty acids on LDL with amino acid residues of the LDL proteins, including tryptophan, arginine, histidine and lysine, and resulting in the neutralisation of the positive charges on the amino acids (Esterbauer, H. et al., 1987, J. Lipid, Res., 28: 495-509; Chen. Q. et al., 1992, Biochem. J., 288: 249-254). Particular modification products of the fatty acids include malondialdehyde (MDA) (formed by the degradation of lipid peroxides) and 4-hydroxynonenal. Modification of LDL can be similarly achieved by glycation of Apo B (for example in poorly controlled diabetics), or by direct oxidation events. Tryptophan can also be modified to give N-formylkynurenine and bityrosine.

The present inventors have now identified peptide fragments of the apoprotein B 100 portion of LDL which are oxidised and which present epitopes which prevent oxidised LDL from being uptaken by the scavenger receptor, thereby preventing the uptake of LDL by the high affinity LDL receptor.

According to the present invention there is provided a molecule having the sequence ALQYKLEGTTR (SEQ ID NO: 1) or a partially modified form thereof or an analogue thereof, lysine 5 being conjugated with MDA, and which inhibits uptake by the high affinity LDL receptor of LDL or a partially modified form thereof. The sequence is residues 3349-3359 of apoprotein B 100 (full Apoprotein B sequence—Knolt, T. J. et al., 1986, Nature, 734-738), lysine 5 corresponding to residue 3353.

Also provided according to the present invention is a molecule having the sequence RLTRKRGLKLA (SEQ ID NO: 2) or a partially modified form thereof or an analogue thereof, lysine 5 being conjugated with MDA, and which inhibits uptake by the high affinity LDL receptor of LDL or a partially modified form thereof. The sequence is residues 3359-3369 of apoprotein B 100, lysine 5 corresponding to residue 3363

Also provided according to the present invention is a molecule having the sequence ALSLSNKFVEG (SEQ ID NO: 3) or a partially modified form thereof or an analogue thereof, lysine 7 being conjugated with MDA, and which inhibits uptake by the high affinity LDL receptor of LDL or a partially modified form thereof. The sequence is residues 3371-3381 of apoprotein B 100, lysine 7 corresponding to residue 3377.

Partial modification of the amino acid sequence may be by way of addition, deletion or substitution of amino acid residues or by other chemical modification, the partially modified molecule inhibiting uptake of LDL by the high affinity LDL receptor. Analogues (for example mimitopes) of the amino acid sequences and the epitopes displayed may be readily produced (Geysen, H. M. et al., 1987, Journal of Immunological Methods, 102: 259-274), the analogues inhibiting uptake by the high affinity LDL receptor of LDL or a partially modified form thereof. Partially modified sequences may be homologues of the sequences from which they were derived.

The conjugated MDA may be either MDA itself or a closely related derivative of MDA, for example an α,β unsaturated aldehyde derivative of MDA.

Modification of LDL may for example be by conjugation with MDA, by glycation or by conjugation with hydroxy alkenals such as 4-hydroxynonenal.

Molecules according to the present invention may be for use as immunogens, e.g. for the production of antibodies (or antigen binding fragments thereof) against them (Harlow, E. and Lane, D., “Antibodies—A Laboratory Manual”, Cold Spring Harbor Laboratory, Cold Spring Harbor Press, New York, 1988). The term “antibody” is used to describe any antigen-binding species, for example an antigen-binding antibody fragment.

The molecules according to the present invention may be for use in a method of treatment or diagnosis of the human or animal body. Particularly, they may be for the treatment of diagnosis of atherosclerosis. Treatment may of course be both prophylactic and therapeutic.

Also provided according to the present invention is the use of a molecule according to the present invention in the manufacture of a medicament for inhibiting uptake by the high affinity LDL receptor of LDL or a partially modified form thereof. Also provided is a method of manufacture of same medicament, comprising the use of a molecule according to the present invention.

Methods of manufacture are well known in the art. For example, a molecule according to the present invention may be provided with a pharmaceutically acceptable carrier, diluent or excipient (Remington's Pharmaceutical Sciences and US Pharmacopeia, 1984, Mack Publishing Company, Easton, Pa., USA), ready for e.g. intravenous injection. Similarly, the dose to give may be readily determined using standard in vitro/in vivo does-response experiments. Culture systems for macrophages are well known and may be readily employed in such experiments.

Also provided according to the present invention is antibody which binds specifically with the molecules of the present invention. Also provided is antibody which binds specifically with the molecules of the present invention for use in a method of treatment or diagnosis of the human or animal body for example detection of modified or conjugated LDL or peptides of same. Molecules, antibodies or antigen binding fragments therof may also be for immunotherapeutic use. Binding agents other than antibodies, which agents bind specifically to the molecules of the present invention may equally be used.

Also provided according to the present invention is the use of antibody according to the present invention in the manufacture of a medicament for inhibiting uptake by the high affinity LDL receptor of LDL or a partially modified form thereof. Also provided is a method of manufacture of same medicament, comprising the use of antibody according to the present invention. A particular use of the antibody is in the treatment or diagnosis of atherosclerosis, treatment being both prophylactic and therapeutic.

Also provided according to the present invention is the use of a molecule according to the present invention in a diagnostic test method for antibody specific against oxidised LDL which will cause uptake by the high affinity LDL receptor of LDL or a partially modified form thereof. Also provided is the use of antibody according to the present invention in a diagnostic test method for oxidised LDL which will cause uptake by the high affinity LDL receptor of LDL or a partially modified for thereof.

Also provided is a diagnostic test method for oxidised LDL which will cause uptake by the high affinity LDL receipt of LDL or a partially modified form thereof, comprising the steps of:

i) reacting an antibody according to the presention invention with a sample;

ii) detecting an antibody-antigen binding reaction; and

iii) correlating detection of the antibody-antigen binding reaction with the presence of oxidised LDL which will cause uptake by the high affinity LDL receptor of LDL or a partially modified form thereof.

Also provided is a diagnostic test method for antibody specific against oxidised LDL which will cause uptake by the high affinity LDL receptor of LDL or a partially modified form thereof, comprising the steps of:

i) reacting a molecule according to the present invention with a sample;

ii) detecting an antibody-antigen binding reaction; and

iii) correlating detection of the antibody-antigen binding reaction with the presence of antibody specific against oxidised LDL which will cause uptake by the high affinity LDL receptor of LDL or a partially modified form thereof.

Also provided according to the present invention is a diagnostic test method for modified or conjugated LDL or peptides of the same which will not be taken up by the high affinity of LDL receptor, comprising the steps of:

i) reacting an antibody or antigen binding fragment specific to a molecule according to the present invention with a sample;

ii) detecting an antibody-antigen binding reaction; and

iii) correlating detection of the antibody-antigen binding reaction with the presence of oxidised or conjugated LDL or peptides of the same which will not be taken up by the high affinity LDL receptor.

The sample may be a patient plasma sample.

The plasma may contain antigen representing various types of oxidation and conjugation.

Molecules and antibody according to the present invention may be used to quantitatively standardise results obtained from such diagnostic tests, or may be used as reagents in the tests. They may have therapeutic benefit as antagonists and could prevent foam cell formation.

Diagnostic test methods may include ELISA (enzyme-linked immunosorbent assay), for example antigen capture ELISA or competitive ELISA, immunoturbidimetry or dip-stick assays (WO 88/08534).

In order to test for specific conditions and causes of oxidation of LDL (for example oxidation caused by hyperglycaemia, hypercholesterolaemia, systemic lupus erythematosis (SLE) or hereditary conditions, molecules according to the present invention modified by for example glycation or conjugation with MDA may be used as appropriate, as may antibody specific to the molecules.

Also provided according to the present invention is a diagnostic test kit for performing a diagnostic test method according to the present invention. Such a kit may include instructions for its use (i.e. for performing an appropriate diagnostic test method according to the present invention).

The invention will be further apparent from the following description, with reference to the several figures of the accompanying drawings, which show, by way of example only, methods of detection of oxidised or conjugated LDL and of peptides of same. Of the figures:

FIG. 1 shows a schematic representation of an LDL particle and Apo B epitopes. The location of Apo B fragment responsible for LDL binding to the high affinity LDL receptor is shown as the amino acid residues 3441-3569 defined by monoclonal antibody Mb47 (Knolt, T. J. et al., supra). Mb47 blocks LDL binding to the high affinity LDL receptor. T₂T₃ suggests the boundary of thrombolytic peptides;

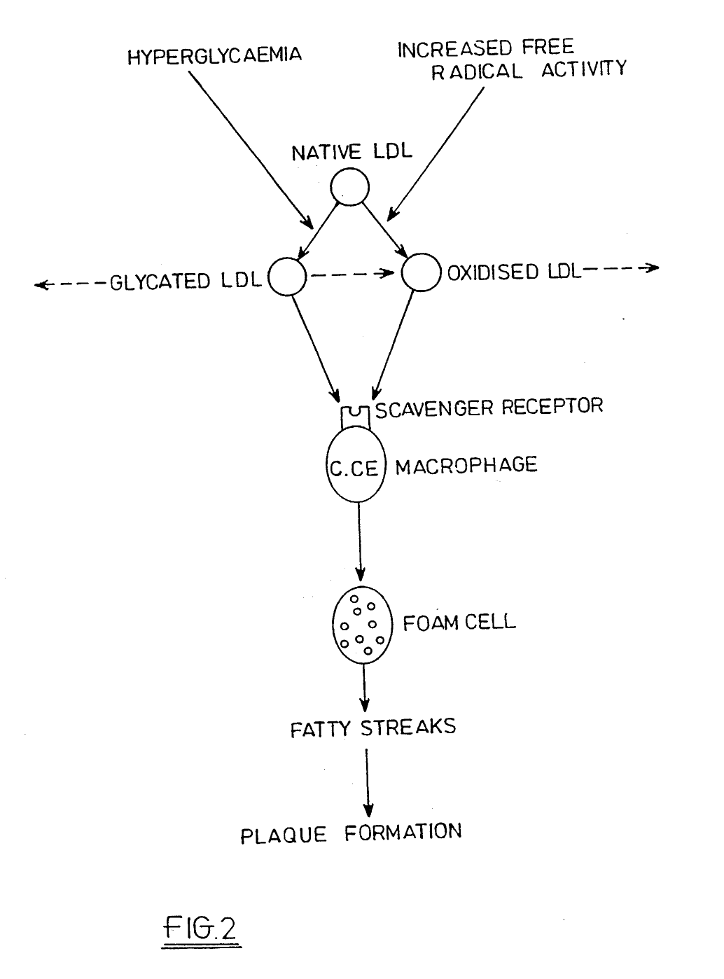

FIG. 2 shows the common mechanism of modification of LDL through glycation and conjugation with MDA. Increased free radical activity caused by non-enzymic glycosylation, the polyol pathway, reduced antioxidant reserves and activated neutrophils etc. causes lipid peroxides, lipid peroxy radicals, lipid alkoxyl radicals and aldehydes to convert native LDL to oxidised LDL, causing endothelial damage amd smooth muscle cell damage. Glycated LDL results in increased platelet aggregation, increased covalent binding to vascular matrix proteins, and endothelial damage;

FIG. 3 shows the conjugation of MDA with lysine; and

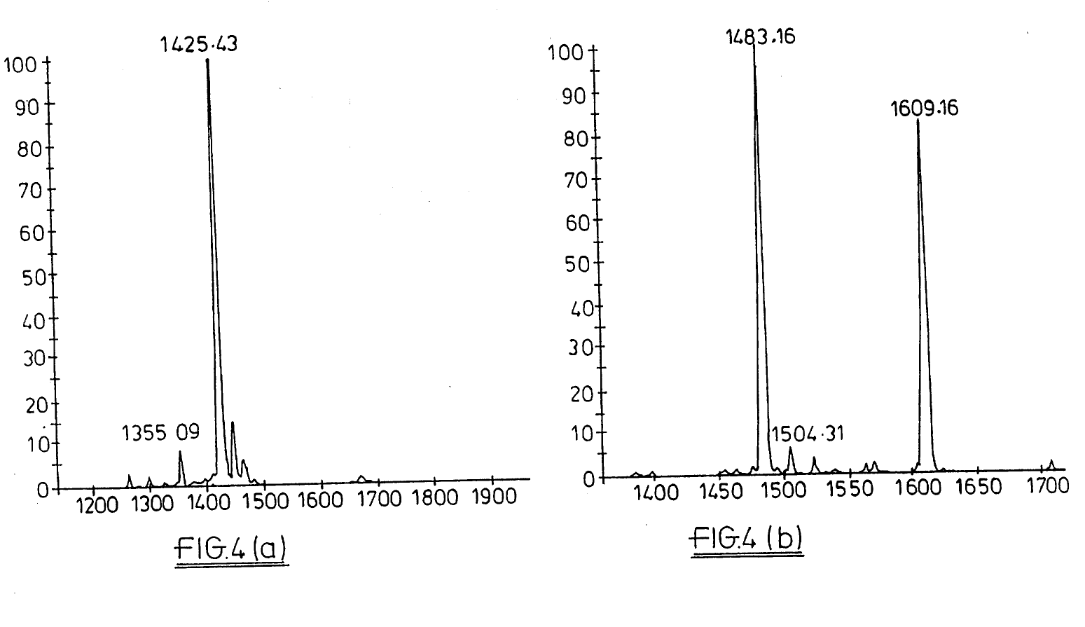

FIG. 4 shows a comparison of peptide ALQYKLEGTTR (SEQ ID NO: 1) before (a) and after (b) conjugation with MDA. X axis shows the mass/charge ratio; Y axis shows relative abundance. Numbered peaks are at (FIG. 4a) 1355.09 and 1425.43 and (FIG. 4b) at 1483.16, 1504.31 and 1609.16 on the X-axis.

### EXPERIMENTAL

The peptide sequence ALQYKLEGTTR (SEQ ID NO: 1) was synthesised using standard methods and conjugated with MDA. FIG. 4 shows the results of a mass spectrometry plot of the peptide before and after conjugation. Molecular weights of products are consistent with peak 1 (FIG. 4(b)) being conjugated with a single molecule of MDA and peak 2 (FIG. 4(b)) being conjugated to a dimeric form of MDA.

Antigen Capture ELISA

Antibody specific against an antigen is coated onto an ELISA plate and used to capture antigen from patient plasma—either total plasma or LDL fractions are used. Binding is then detected using a second antibody specific against the antigen followed by an enzyme-conjugated anti-immunoglobulin for colorimetric detection.

Competitive ELISA

An ELISA plate is coated with either MDA-conjugated ALQYKLEGTIR (SEQ ID NO: 1) peptides or with oxidised or conjugated LDL or peptides of the same which will not be taken up by the high affinity LDL receptor. Serial dilutions of patient serum (for example total plasma or LDL fractions can be added) are added together with antibody of fixed dilution. Binding of antibody to coating antigen is detected using enzyme-conjugated anti-immunoglobulin, the extent of binding reducing as the concentration of antigen in the plasma increases. Results are standardised by producing a standard curve using MDA-conjugated ALQYKLEGTTR (SEQ ID NO: 1) peptides.

Immunoturbidimetry

Latex beads are coated with antibody specific to MDA-conjugated ALQYKLEGTTR (SEQ ID NO: 1) peptides, and the beads mixed with patient sera. The aggregation of the beads due to antibody cross-linking in the presence of specific antigen is analysed in autoanalyzers using immunoturbidimetry detection systems.

The procedures described above are used in diagnosis, for example in ELISA, immunoturbidimetry or dip-stick assays and test kits.

1. A molecule having the sequence of SEQ ID NO: 1 or a partially modified form thereof or an analogue thereof, lysine 5 being conjugated with MDA, and which inhibits uptake by the high affinity LDL receptor of LDL or a partially modified form thereof.

2. A molecule having the sequence of SEQ ID NO: 2 or a partially modified form thereof or an analogue thereof, lysine 5 being conjugated with MDA, and which inhibits uptake by the high affinity LDL receptor of LDL or a partially modified form thereof.

3. A molecule having the sequence of SEQ ID NO: 3 or a partially modified form thereof or an analogue thereof, lysine 7 being conjugated with MDA, and which inhibits uptake by the high affinity LDL receptor or LDL or a partially modified from thereof.

4. A molecule according to any one of claims 1 to 3, LDL being modified by conjugation with MDA.

5. A molecule according to any one of claims 1 to 3, LDL being modified by glycation.

6. A molecule according to any one of claims 1 to 3, LDL being modified by conjugation with hydroxy alkenals.

7. A molecule according to claim 6, the conjugation being with 4-hydroxynonenal.

8. A molecule according to any one of the preceding claims, being an immunogen.

9. A molecule according to any one of the preceding claims for use in a method of treatment or diagnosis of the human or animal body.

10. The use of a molecule according to any one of claims 1-8 in the manufacture of a medicament for inhibiting uptake by the high affinity LDL receptor of LDL or a partially modified form thereof.

11. A method of manufacture of a medicament for inhibiting the uptake by the high affinity LDL receptor of LDL or a partially modified form thereof, comprising the use of a molecule according to any one of claims 1-8.

12. Antibody which binds specifically with a molecule according to any one of claims 1-8.

13. Antibody according to claim 12 for use in a method of treatment or diagnosis of the human or animal body.

14. The use of antibody according to claim 12 in the manufacture of a medicament for inhibiting uptake by the high affinity LDL receptor of LDL or a partially modified form thereof.

15. A method of manufacture of a medicament for inhibiting uptake by the high affinity LDL receptor of LDL or a partially modified form thereof, comprising the use of antibody according to claim 12.

16. The use of a molecule according to any one claims 1-7 in a diagnostic test method for antibody specific against oxidised LDL which will cause uptake by the high affinity LDL receptor of LDL or a partially modified form thereof.

17. The use of antibody according to claim 12 in a diagnostic test method for oxidised LDL which will cause uptake by the high affinity LDL receptor of LDL or a partially modified form thereof.

18. A diagnostic test method for oxidised LDL which will cause uptake by the high affinity LDL receptor of LDL or a partially modified form thereof, comprising the steps of:
i) reacting an antibody according to claim 12 with a sample;
ii) detecting an antibody-antigen binding reaction; and
iii) correlating detection of the antibody-antigen binding reaction with the presence of oxidised LDL which will cause uptake by the high affinity LDL receptor of LDL or a partially modified form thereof.

19. A diagnostic test method for antibody specific against oxidised LDL which will cause uptake by the high affinity LDL receptor of LDL or a partially modified form thereof, comprising the steps of:
i) reacting a molecule according to any one of claims 1-7 with a sample;
ii) detecting an antibody-antigen binding reaction; and
iii) correlating detection of the antibody-antigen binding reaction with the presence of antibody specific against oxidised LDL which will cause uptake by the high affinity LDL receptor of LDL or a partially modified form thereof.

20. A diagnostic test method for modified or conjugated LDL or peptides of the same which will not be taken up by the high affinity LDL receptor, comprising the steps of:
i) reacting an antibody or antigen binding fragment specific to a molecule according to any one of claims 1 to 7 with a sample;
ii) detecting an antibody-antigen binding reaction; and
iii) correlating detection of the antibody-antigen binding reaction with the presence of modified or conjugated LDL or peptides of the same which will not be taken up by the high affinity LDL receptor.

21. A diagnostic test method according to any one of claims 18-20, the sample being a patient plasma sample.

22. A diagnostic test kit for performing a diagnostic test method according to any one of claims 18-21.
